(19) United States
(12) Patent Application Publication
(10) Pub. No.: US 2015/0094534 A1
(43) Pub. Date: Apr. 2, 2015

(54) ENDOSCOPE AND METHOD FOR MANUFACTURING THE SAME

(71) Applicant: FUJIFILM CORPORATION, Tokyo (JP)

(72) Inventor: Hideyuki YAMADA, Ashigarakami-gun (JP)

(73) Assignee: FUJIFILM CORPORATION, Tokyo (JP)

(21) Appl. No.: 14/499,718

(22) Filed: Sep. 29, 2014

(30) Foreign Application Priority Data
Sep. 30, 2013 (JP) 2013-204750

(51) Int. Cl. A61B 1/012; A61B 1/00; A61B 1/05

(52) U.S. Cl. CPC A61B 1/012; A61B 1/05; A61B 1/00018; A61B 1/0011
USPC 600/110; 29/592.1

(57) ABSTRACT

The present invention aims to provide an endoscope and its manufacturing method enabling to reduce a diameter of the endoscope required to have airtightness and facilitate a connecting work between a cable and a connector. A first tube body and a second tube body constituting an airtight container are relatively moved to extend and contract, so that it is possible to extend cables from a proximal end part of the second tube body by contracting the container, thereby enabling a connecting work between the cables and an airtight connector. After the completion of connecting work between the cables and the airtight connector, it is possible to store an extra length of the cables in the second tube body as well as to allow the airtight connector to engage with the proximal end part of the second tube body by extending the first tube body and the second tube body.

### CROSS-REFERENCE TO RELATED APPLICATIONS

The patent application claims priority under 35 U.S.C. §119 to Japanese Patent Application No. 2013-204750, filed on Sep. 30, 2013. Each of the above application(s) is hereby expressly incorporated by reference, in its entirety, into the present application.

### BACKGROUND OF THE INVENTION

1. Field of the Invention

The present invention relates to an endoscope and a method for manufacturing an endoscope, and more particularly to an endoscope and a method for manufacturing an endoscope for which high airtightness is required.

2. Description of the Related Art

An endoscope used for surgery or endoscopy requires sterilization treatment to prevent infection among patients. In recent years, a sterilization method, with which an endoscope is put in a high pressure steam sterilizer such as an autoclave to be sterilized by high pressure steam, is gradually becoming the mainstream.

Conventionally, there is proposed a technique for forming a distal end of an endoscope, in which an optical unit and an imaging device unit are stored, so that the distal end has an airtight structure that can cope with the autoclave (Japanese Patent Application Laid-Open No. 2002-301016).

An endoscope described in Japanese Patent Application Laid-Open No. 2002-301016 includes a lens unit and a solid imaging element, which are provided in a pipe-shaped element frame as shown in FIG. 2 in Japanese Patent Application Laid-Open No. 2002-301016. A lens on the tip of the lens unit is fixed to a distal end part of the element frame in an airtight manner, and a connector is fixed to a proximal end part of the element frame in an airtight manner, thereby allowing the element frame to have an airtight structure. The solid imaging element is electrically connected to a hybrid IC (hybrid integrated circuit) through an element lead pin, and the hybrid IC is fixed to the connector through a connection terminal. That is, the solid imaging element is fixed to the connector through the hybrid IC.

An endoscope of another embodiment shown in FIG. 13 in Japanese Patent Application Laid-Open No. 2002-301016 includes two bodies of a first element frame in which a lens unit is provided and a second element frame in which a solid imaging element is provided. The first element frame and the second element frame are joined with each other by metal joining or the like in an airtight manner.

The first element frame and the second element frame are relatively movable in an axial direction, so that it is possible to perform focus adjustment by changing relative positions of the first element frame and the second element frame before the joining.

### SUMMARY OF THE INVENTION

In the endoscope described in Japanese Patent Application Laid-Open No. 2002-301016, the solid imaging element is connected to the connector through the element lead pin and the hybrid IC, and has a structure in which cables are not arranged between the solid imaging element (or the hybrid IC) and the connector. Thus, a connecting work between cables that connect the hybrid IC and the connector is unnecessary; however, the invention described in Japanese Patent Application Laid-Open No. 2002-301016 is not applicable to an endoscope of a type in which a hybrid IC is connected to a connector by cables.

Furthermore, in the invention described in Japanese Patent Application Laid-Open No. 2002-301016, since the solid imaging element is connected to the connector through the element lead pin and the hybrid IC, the solid imaging element and the connector are fixed to the element frame so as to be parallel to each other. Thus, the solid imaging element to be applied to the endoscope described in Japanese Patent Application Laid-Open No. 2002-301016 is limited to a longitudinally mounted solid imaging element that is provided in a direction orthogonal to a longitudinal direction of the element frame (endoscope). Therefore, the invention described in Japanese Patent Application Laid-Open No. 2002-301016 has a problem that it is not applicable to a transversely mounted imaging device which is advantageous for reducing a diameter of an endoscope.

The present invention has been made in light of the above-mentioned circumstances, and the present invention aims to provide an endoscope and a method for manufacturing an endoscope capable of reducing a diameter of the endoscope required to have airtightness and facilitating a connecting work for connecting cables to a connector.

In order to achieve the object above, an endoscope according to one aspect of the present invention includes: a first tube body that is provided with an optical window joined to a distal end thereof in an airtight manner, and that stores an optical unit and an imaging device unit; a second tube body that is movable by sliding on an inner circumferential surface or an outer circumferential surface of the first tube body; and an airtight connector that is joined to a proximal end part of the second tube body in an airtight manner, and that is connected to cables extending from the imaging device unit, wherein the first tube body and the second tube body are joined together in an airtight manner at least after the cables and the airtight connector are connected.

According to one aspect of the present invention, the first tube body and the second tube body constituting an airtight container can extend and contract by relatively moving to each other. When the first tube body and the second tube body are relatively moved so that a length from the distal end of the first tube body to the proximal end part of the second tube body is shortened, it is possible to extend the cables from proximal end part of the second tube body, thereby enabling a connecting work for connecting the cables and the airtight connector. After the connecting work for connecting the cables and the airtight connector is finished, it is possible to store an extra length of the cables in the second tube body by relatively moving the first tube body and the second tube body so that the length from the distal end of the first tube body to the proximal end part of the second tube body is increased, as well as to allow the airtight connector to engage with the proximal end part of the second tube body. The optical window to be joined to the distal end of the first tube body in an airtight manner is not limited to a transparent parallel flat plate, but a plate serving as a lens is available.

In an endoscope according to another aspect of the present invention, it is preferable that the cables have a length corresponding to a distance from the imaging device unit to the airtight connector. Accordingly, it is possible to minimize inner diameters of the first and second tube bodies for allowing the cables to be stored in the tube bodies, so that a diameter of the endoscope can be reduced.

In an endoscope according to yet another aspect of the present invention, it is preferable that one of the first tube body and the second tube body has a fitting part in which the other of the first tube body and the second tube body is fitted with a fixed gap, and the fitting part has a length in which an extra length of the cables is adjustable. Accordingly, in a case where the extra length of the cables is stored in the second tube body by relatively moving the first tube body and the second tube body so that length from the distal end of the first tube body to the proximal end part of the second tube body is increased after the connecting work between the cables and the airtight connector is finished, even if there are variations in the extra length of the cables, it is possible to join the first and second tube bodies with the fitting part having the fixed gap at the time of joining a part between the first and second tube bodies in an airtight manner, because an end part of the first tube body or the second tube body is brought into contact with an inner circumferential surface or an outer circumferential surface the other tube body within a range of the length of the fitting part. If the length of the fitting part is so long that resistance at the time of sliding is increased, it is impossible to smoothly perform extension and contraction movement of the first tube body and the second tube body. Thus, the fitting part cannot have a length longer than a length in which the extension and contraction movement of the first tube body and the second tube body can be smoothly performed.

In an endoscope according to yet another aspect of the present invention, it is preferable that the airtight connector includes a plurality of pins penetrating through a connector body in an airtight manner, and that the cable and the airtight connector are electrically connected through pipe-shaped conductive members in which the pins are fitted, that is, one end of each of the cables is inserted into one end of each of the pipe-shaped conductive members to be fixed, and each of the pins of the airtight connector is inserted into the other end of each of the conductive members, whereby it is possible to easily connect the cables to the airtight connector.

In an endoscope according to yet another aspect of the present invention, it is preferable that a maximum movement amount by which the first tube body and the second tube body are relatively movable is twice or more as large as a length from the airtight connector to an end part of each of the conductive members connected to the airtight connector on a side connected to each of the cables, in order to secure flexibility of the conductive members when each of the pins of the airtight connector is inserted into each of the pipe-shaped conductive members is inserted.

In an endoscope according to yet another aspect of the present invention, it is preferable that the optical unit includes a folded optical system, and the imaging device unit is arranged to be parallel to a longitudinal direction of the first tube body. For the imaging device unit, a transversely mounted type arranged to be parallel the longitudinal direction of the first tube body is used so that the diameter of the endoscope can be reduced as compared with a longitudinally mounted type.

In an endoscope according to yet another aspect of the present invention, it is preferable that the first tube body includes a third tube body that is provided with the optical window fixed to a distal end thereof in an airtight manner, and that stores the optical unit and the imaging device unit; and a fourth tube body that is joined to the third tube body in an airtight manner. Thus, it is possible to shorten the length of the third tube body that stores the optical unit and the imaging device unit by dividing the first tube body into two tube bodies of the third tube body and the fourth tube body. Accordingly, an operation of providing the optical unit and the imaging device unit in the third tube body is facilitated. In addition, the fourth tube body is joined to the third tube body so that a movement amount by which the fourth tube body and the second tube body can be relatively moved can be secured in a part between the fourth tube body and the second tube body.

A method for manufacturing an endoscope according to yet another aspect of the present invention includes: preparing a first tube body storing an optical unit and an imaging device unit, a second tube body movable by sliding on an inner circumferential surface or an outer circumferential surface of the first tube body, and an airtight connector to which cables extending from the imaging device unit are connected; extending the cables from a proximal end part of the second tube body by relatively moving the first tube body and the second tube body so that a length from a distal end of the first tube body to the proximal end part of the second tube body is shortened; electrically connecting the cables extending from the proximal end part of the second tube body and the airtight connector; engaging the airtight connector with the proximal end part of the second tube body by relatively moving the first tube body and the second tube body so that the length from the distal end of the first tube body to the proximal end part of the second tube body is increased after the cables and the airtight connector are connected; joining the optical window to the distal end of the first tube body in an airtight manner; joining the first tube body and the second tube body in an airtight manner; and joining the proximal end part of the second tube body and the airtight connector in an airtight manner.

Although it is possible to perform joining operations of joining the optical window to the distal end of the first tube body in an airtight manner, joining the first tube body and the second tube body in an airtight manner, and joining the proximal end part of the second tube body and the airtight connector in an airtight manner in any order and timing, it is necessary that the joining the first tube body and the second tube body in an airtight manner, and joining the proximal end part of the second tube body and the airtight connector in an airtight manner, are performed at least after the cables and the airtight connector are connected.

In a method for manufacturing an endoscope according to yet another aspect of the present invention, it is preferable that after the cables and the airtight connector are connected, the first tube body and the second tube body are relatively moved so that the length from the distal end of the first tube body to the proximal end part of the second tube body is increased, and the airtight connector is joined to the proximal end part of the second tube body, and that the cables have a length corresponding to a distance from the imaging device unit to the airtight connector at a time when the airtight connector is joined to the proximal end part of the second tube body.

In a method for manufacturing an endoscope according to yet another aspect of the present invention, it is preferable that the airtight connector includes a plurality of pins penetrating through a connector body in an airtight manner, and that, in the step of electrically connecting the cables and the airtight connector, the cables and the airtight connector are electrically connected through pipe-shaped conductive members in which the pins are fitted.

In a method for manufacturing an endoscope according to yet another aspect of the present invention, it is preferable that a maximum movement amount by which the first tube body and the second tube body are relatively movable is twice or more as large as a length of a rigid part of the pins of the airtight connector and the conductive members.

In a method for manufacturing an endoscope according to yet another aspect of the present invention, it is preferable that the first tube body includes a third tube body and a fourth tube body, and that the method further includes: storing the optical unit and the imaging device unit in the third tube body; fitting the fourth tube body in the third tube body; and joining the third tube body and the fourth tube body in an airtight manner.

According to the present invention, since the first tube body storing the optical unit and the imaging device unit and the subsequent second tube body are configured to be relatively movable (extendable), it is possible to extend the cables from the proximal end part of the second tube body by relatively moving the first tube body and the second tube body so that the length from the distal end of the first tube body to the proximal end part of the second tube body is shortened, thereby enabling the connecting work between the cables and the airtight connector to be facilitated. In addition, it is possible to store an extra length of the cables in the second tube body by relatively moving the first tube body and the second tube body so that the length from the distal end of the first tube body to the proximal end part of the second tube body is increased after the connecting work between the cable and the airtight connector is completed, whereby it is possible to minimize a space for storing the cables so that a diameter of the endoscope can be reduced.

### BRIEF DESCRIPTION OF THE DRAWINGS

FIG. 1 is a whole configuration diagram showing a first embodiment of the endoscope according to the present invention;

FIG. 2 is an external view of a distal end rigid part of the endoscope shown in FIG. 1 showing a contracted state;

FIG. 3 is an external view of the distal end rigid part of the endoscope shown in FIG. 1 showing an extended state;

FIG. 4 is a cross-sectional view of an enlarged main section of the distal end rigid part of the endoscope shown in FIG. 2;

FIG. 5 is a cross-sectional view of an enlarged main section of the distal end rigid part of the endoscope shown in FIG. 3;

FIG. 6 is a front view of the airtight connector;

FIG. 7 is a cross-sectional view of the airtight connector shown in FIG. 6 taken along the line 7-7;

FIG. 8 is a perspective view showing a state in which the cables and the airtight connector are connected;

FIGS. 9A, 9B, 9C, and 9D show a procedure of connecting the cable and the airtight connector;

FIG. 10 is an external view showing a second embodiment of the endoscope according to the present invention in a state in which the distal end rigid part of an insertion section of the endoscope is contracted;

FIG. 11 is an external view showing a second embodiment of the endoscope according to the present invention in a state in which the distal end rigid part of the insertion section of the endoscope is extended;

FIG. 12 is a cross-sectional view of an enlarged main section of the distal end rigid part of the endoscope shown in FIG. 10; and

FIG. 13 is a cross-sectional view of an enlarged main section of the distal end rigid part of the endoscope shown in FIG. 11.

### DETAILED DESCRIPTION OF THE PREFERRED EMBODIMENTS

Referring to accompanying drawings, preferable embodiments of the endoscope and the method for manufacturing an endoscope according to the present invention will be described.

### First Embodiment

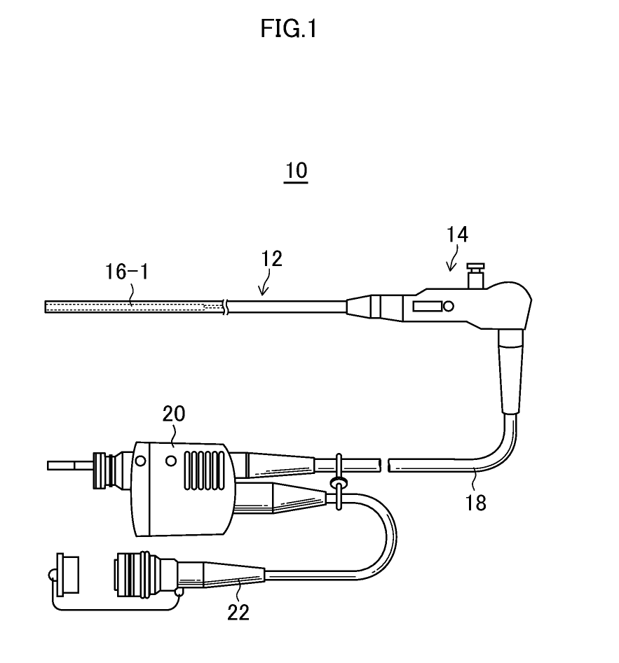

FIG. 1 is a whole configuration diagram showing a first embodiment of the endoscope according to the present invention.

An endoscope 10 shown in FIG. 1 is an electronic endoscope applied to surgery, and includes an insertion section 12 to be inserted in a body cavity of a patient, and an operation section 14 to be held by a surgeon. The insertion section 12 includes a distal end rigid part 16-1 that stores an optical unit, an imaging device unit, and the like, which will be described. The distal end rigid part 16-1 is provided inside an exterior case of the insertion section 12.

A universal cable 18 is connected to the operation section 14, and a light guide (LG) connector 20 is provided at a tip of the universal cable 18. The LG connector 20 is detachably coupled to a light source device (not shown). Accordingly, illumination light is emitted from the light source device to the distal end of the insertion section 12 through a light guide (not shown) in the endoscope 10 so that it is possible to illuminate the inside of the body cavity. Furthermore, a video connector 22 is connected to the LG connector 20, and the video connector 22 is detachably coupled to a processor (not shown) for performing image processing and the like.

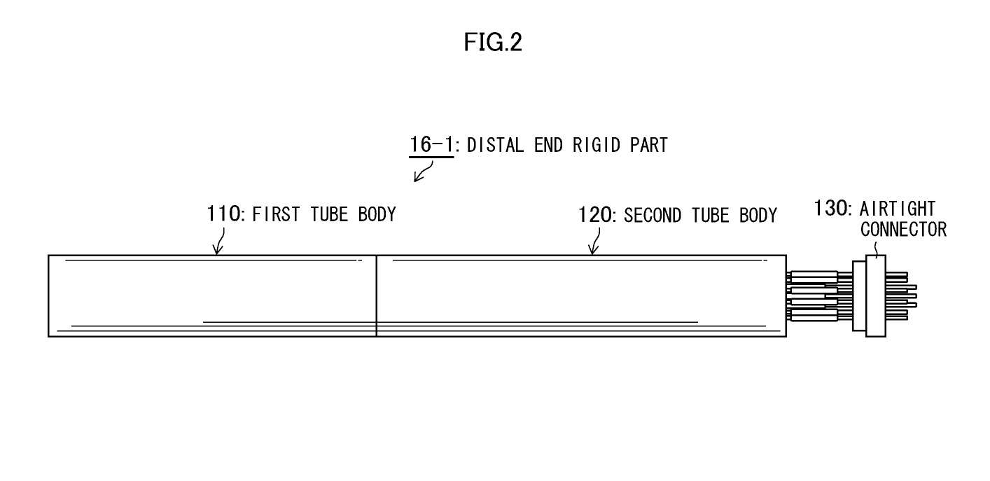

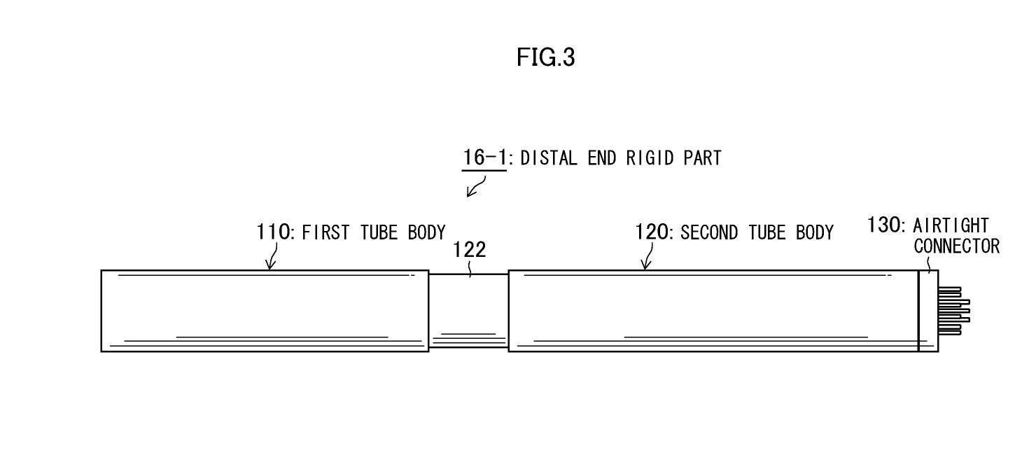

FIGS. 2 and 3 are external views of the distal end rigid part 16-1 of the endoscope 10 showing a state in which the distal end rigid part 16-1 is contracted and a state in which the distal end rigid part 16-1 is extended, respectively.

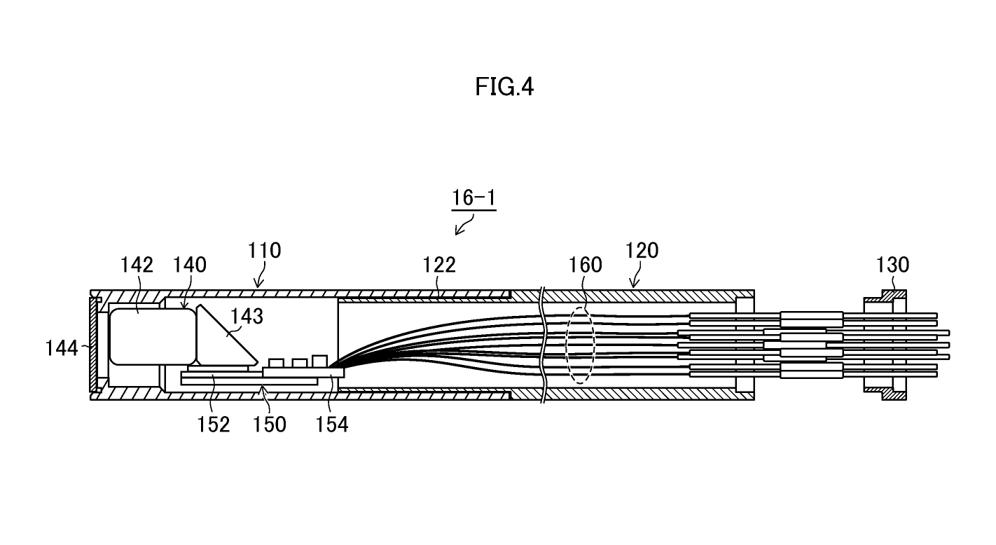

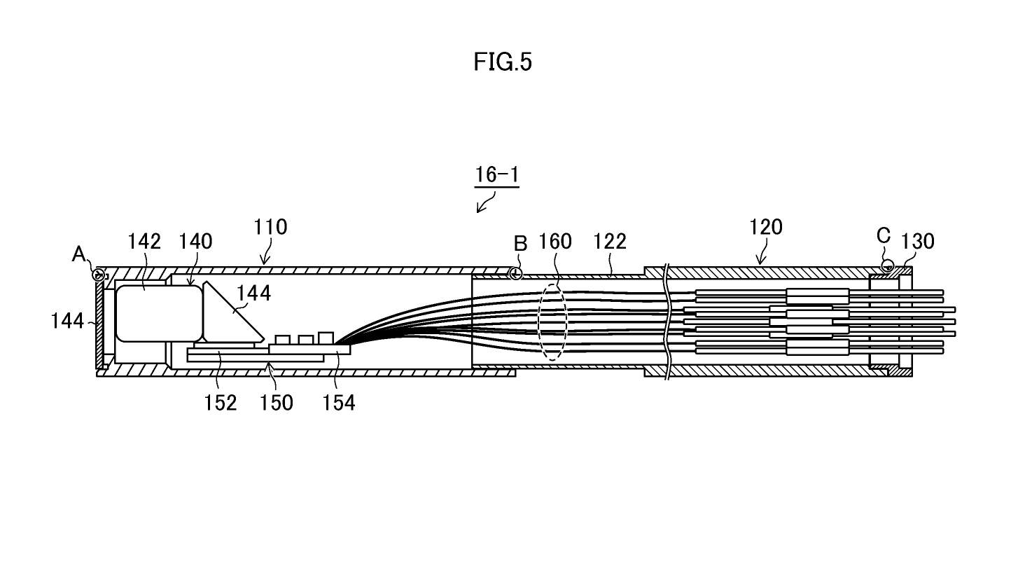

FIGS. 4 and 5 are cross-sectional views showing enlarged main sections of the distal end rigid part 16-1 shown in FIGS. 2 and 3, respectively.

As shown in FIGS. 2 to 5, the distal end rigid part 16-1 is mainly composed of a first tube body 110 that stores an optical unit 140 and an imaging device unit 150, a second tube body 120 that is joined to the first tube body 110, and an airtight connector 130.

The first tube body 110 and the second tube body 120 are main members constituting an airtight container, and each of the first tube body 110 and the second tube body 120 is a metal tube made of stainless steel (SUS). The tube bodies can be formed of kovar (trademark), titanium or the like, instead of the SUS.

A slide part 122 (a part in which a diameter of an outer circumferential surface thereof is smaller than a diameter of an outer circumferential surface of a part other than the slide part 122) is formed in a distal end side of the second tube body 120, so that the outer circumferential surface of the slide part 122 is slidably fitted into an inner circumferential surface of the first tube body 110. Accordingly, the first tube body 110 and the second tube body 120 can be extended and contracted by relatively moving to each other, that is, the first tube body 110 and the second tube body 120 form a telescopic structure such as a telescope.

In the first tube body 110, the optical unit 140 and the imaging device unit 150 are stored, and fixed to the inside of the first tube body 110. The optical unit 140 includes an objective lens 142 and a prism (folded optical system) 143. The imaging device unit 150 includes an imaging element 152 such as a charge coupled device (CCD), a complementary metal oxide semiconductor (CMOS), and the like, and a circuit board 154 on which the imaging element 152, a driving circuit component, an integrated circuit component, and the like are mounted.

A picture imaged by the objective lens 142 is imaged on a light receiving surface of the imaging element 152 through the prism 143, and then is converted into an electric signal. The optical unit 140 including the prism 143 is used as above so that a transversely mounted type of an imaging device unit, which can be arranged parallel to a longitudinal direction (an optical axis direction of the objective lens 142) of the first tube body 110, is used for the imaging device unit 150. The transversely mounted type is advantageous for reducing a diameter of the endoscope 10 as compared with a longitudinally mounted type.

In the distal end of the first tube body 110, the optical window 144 is joined in an airtight manner. The optical window 144 is a parallel flat plate formed of transparent sapphire, quartz, and the like, and a metal film is evaporated (metalized) onto a surface of the optical window to secure a joining property, and is joined to the distal end of the first tube body 110 by soldering in an airtight manner. In the present embodiment, although the optical window 144 is a transparent parallel flat plate, a part of a lens included in the optical unit 140 may be used.

The other ends of the cables (seventeen cables in the present embodiment) 160 wired to the imaging device unit 150 (the circuit board 154) are connected to the airtight connector 130 attached to a proximal end part of the second tube body 120.

It is preferable that a connecting work between the cables 160 and the airtight connector 130 is performed in a state in which the first tube body 110 and the second tube body 120 are most contracted. Thus, it is possible to increase an extra length of the cables 160 extending outside from the proximal end of the second tube body 120 by relatively moving the first tube body 110 and the second tube body 120 so that a length (overall length) from the distal end of the first tube body 110 to the proximal end of the second tube body 120 is most shortened. Accordingly, flexibility of the cables 160 at the time of the connecting work is secured to enable the connecting work.

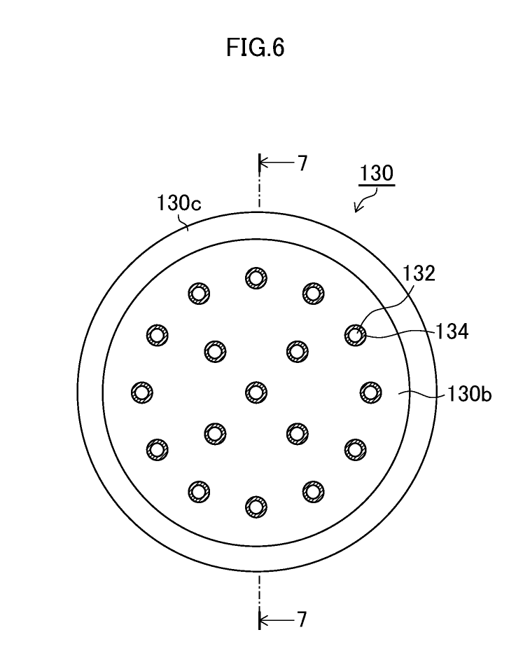

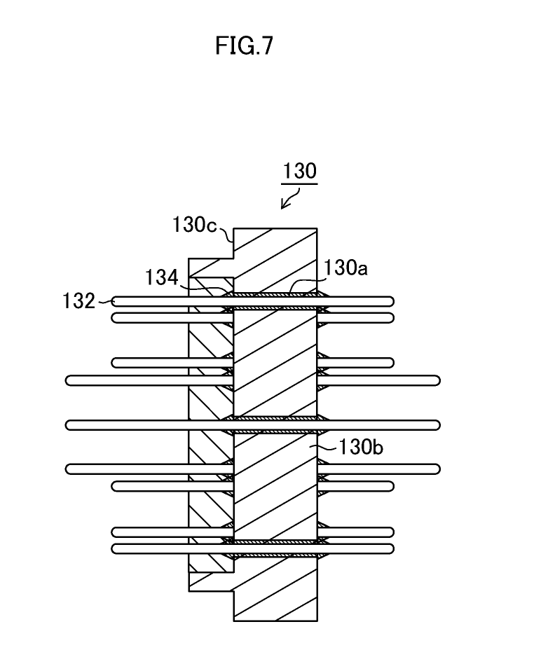

FIG. 6 is a front view of the airtight connector 130, and FIG. 7 is a sectional view of the airtight connector 130 shown in FIG. 6 taken along the line 7-7.

As shown in FIGS. 6 and 7, the airtight connector 130 is composed of: a connector body (base) 130b made of kovar, in which through-holes 130a are formed; pins 132 made of kovar arranged in the through-holes 130a; and sealing glass 134 made of borosilicate glass or the like, which fixes the base 130b and the pins 132 in a mutually insulted manner. The structure enables the airtight connector 130 to maintain airtightness between the base 130b and the pins 132.

The base 130b of the airtight connector 130 is formed into a shape to be inserted and fitted in the proximal end part of the second tube body 120, and has a flange part 130c that is brought into contact with an end face of the proximal end part of the second tube body 120.

The base 130b can be formed of borosilicate glass or the like other than the kovar, and the pins 132 can be formed of copper, brass, or the like other than the kovar. In addition, the sealing glass 134 can be formed of ceramic-based sealant or the like other than the borosilicate glass. A raw material and a structure of the airtight connector 130 are not limited to the embodiment above, so that various materials and structures are applicable.

[Connecting Method of Cables and Airtight Connector]

Next, a connecting method of the cables 160 and the airtight connector 130 will be described.

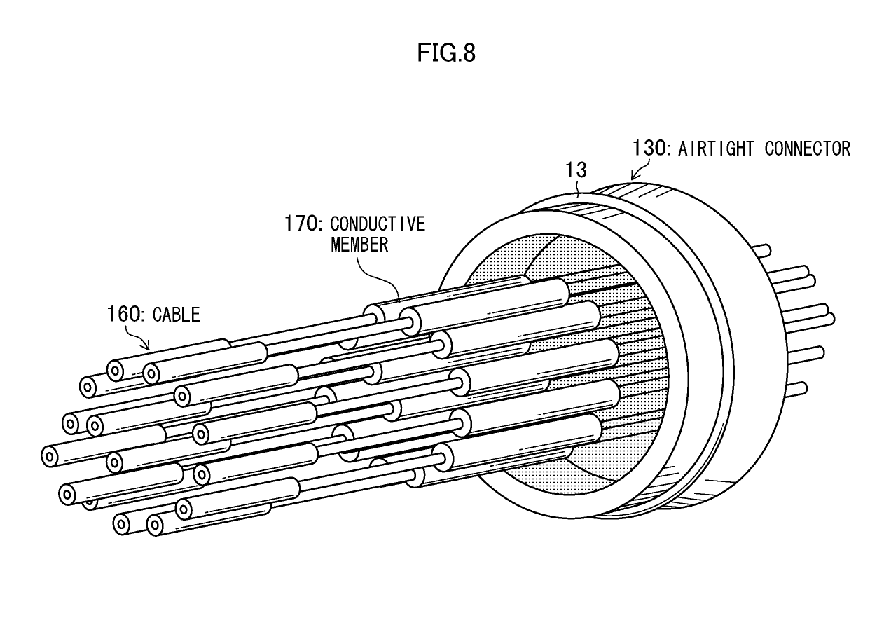

FIG. 8 is a perspective view showing a state in which the cables 160 and the airtight connector 130 are connected.

As shown in FIG. 8, the cables 160 and the airtight connector 130 are electrically connected through pipe-shaped conductive members 170.

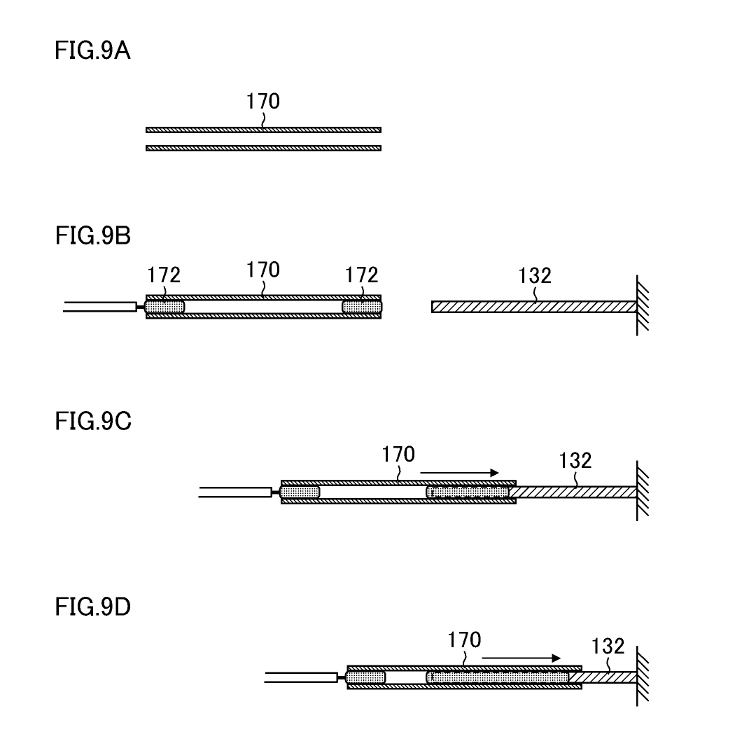

FIGS. 9A, 9B, 9C, and 9D show a procedure of connecting the cable 160 and the airtight connector 130 by using the pipe-shaped conductive member 170.

As shown in FIG. 9A, first the pipe-shaped conductive member 170 is prepared.

Next, the inside of one end of the conductive member 170 is filled with conductive adhesive 172, and then a core wire of the cable 160 is inserted into the inside of the conductive member 170 (FIG. 9B). The conductive adhesive 172 is cured at a room temperature for about 24 hours to connect and fix the core wire to the inside of the conductive member 170.

Subsequently, the inside of the other end of the conductive member 170 to which the cable 160 is connected and fixed is filled with the conductive adhesive 172, and then the conductive member 170 is moved so that the pin 132 is inserted into the other end of the conductive member 170 (FIGS. 9B, 9C, and 9D).

Finally, the conductive adhesive 172 is cured at the room temperature for about 24 hours to connect and fix the pin 132 to the inside of the conductive member 170 (FIG. 9D). As the conductive adhesive 172, Aremco-Bond 525 (trademark) (a heat-resistant temperature of 170° C.) and Aremco-Bond 556 (a heat-resistant temperature of 170° C.), (made by Aremco Products Inc.). Duralco 120 (trademark) (a heat-resistant temperature of 260° C.) (made by Cotronics Corp.), and the like, are available.

Using the conductive adhesive 172 having a high heat-resistance (a heat-resistant temperature of 130° C. or more) enables adhesion to be maintained even in a high temperature environment, thereby making the endoscope 10 applicable to a high pressure and high temperature steam sterilizer (autoclave).

FIGS. 2 and 4 show a state in which the connecting work between the cables 160 and the airtight connector 130 has been performed in a manner as described above. A part of the cables 160 in the state is extended from the proximal end part of the second tube body 120 by a predetermined amount (corresponding to a maximum movement amount by which the first tube body 110 and the second tube body 120 are relatively movable).

From the state, the first tube body 110 and the second tube body 120 are relatively moved so that a length from the distal end of the first tube body 110 to the proximal end part of the second tube body 120 is increased, whereby the airtight connector 130 is fitted to the proximal end part of the second tube body 120 as shown in FIGS. 3 and 5.

Subsequently, as shown in FIG. 5, a fitting part A between the optical window 144 provided with a surface on which a metal film is evaporated and the distal end of the first tube body 110 is sealed by soldering.

Next, the first tube body 110 and the second tube body 120 are joined in an airtight manner. In the embodiment, a fitting part B (whole circumference of a proximal end part of the first tube body 110) in which the first tube body 110 and the second tube body 120 are fitted is sealed by laser welding.

Finally, the proximal end part of the second tube body 120 and the airtight connector 130 are joined in an airtight manner. In the embodiment, a fitting part C (whole circumference of the proximal end part of the second tube body 120) in which the proximal end part of the second tube body 120 and the airtight connector 130 are fitted is sealed by laser welding.

Accordingly, it is possible to maintain airtightness of the inside of the distal end rigid part 16-1 (the first tube body 110 and the second tube body 120) storing the optical unit 140 and the imaging device unit 150, and the endoscope 10 provided with the distal end rigid part 16-1 can cope with autoclave sterilization. The fitting parts may be sealed by not only the laser welding but another metal welding.

Order of sealing operations in three fitting parts A, B, and C is not limited to the order described above. The sealing operation for the fitting part A between the optical window 144 and the distal end of the first tube body 110 may be performed before the connecting work between the cables 160 and the airtight connector 130.

According to the first embodiment of the present invention, as shown in FIGS. 2 and 4, the cables 160 are extended from the proximal end part of the second tube body 120 by relatively moving the first tube body 110 and the second tube body 120 so that the length from the distal end of the first tube body 110 to the proximal end part of the second tube body 120 is shortened, thereby enabling the connecting work between the cable 160 and the airtight connector 130.

As shown in FIGS. 3 and 5, after the connecting work is finished, the extra length of the cables 160 is stored in the second tube body 120 by relatively moving the first tube body 110 and the second tube body 120 so that the length from the distal end of the first tube body 110 to the proximal end part of the second tube body 120 is increased so that it is possible to wire the plurality of (seventeen cables in the present embodiment) cables 160 over a section from the imaging device unit 150 (circuit board 154) to the airtight connector 130 in a substantially linear (including a case of being bent) state, whereby it is possible to minimize an inner diameter (an inner diameter for storing the cables 160) of each of the first tube body 110 and the second tube body 120 so that a diameter of the endoscope 10 can be reduced. That is, the cables 160 are made to have a length corresponding to a distance from the imaging device unit 150 to the airtight connector 130 so that it is possible to wire the cables 160 over the section from the imaging device unit 150 to the airtight connector 130 in a substantially linear state.

### Second Embodiment

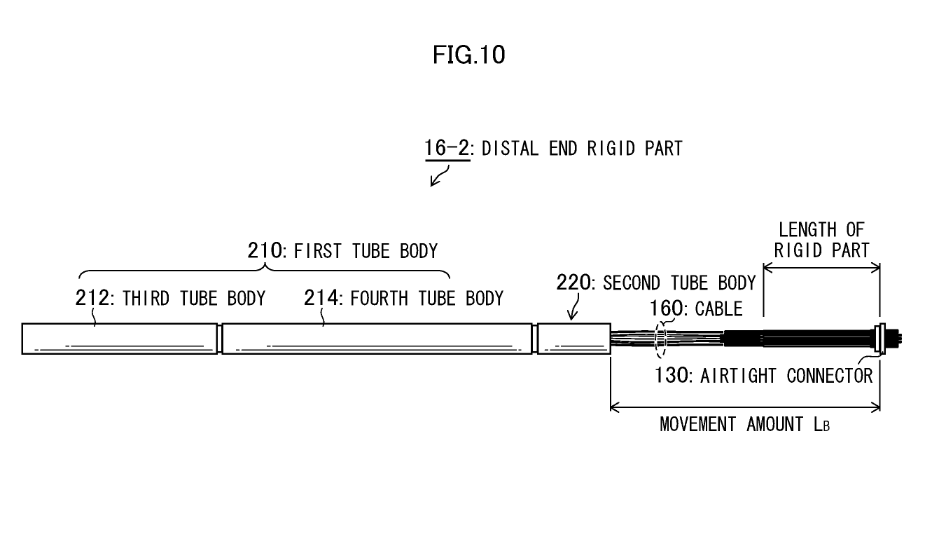

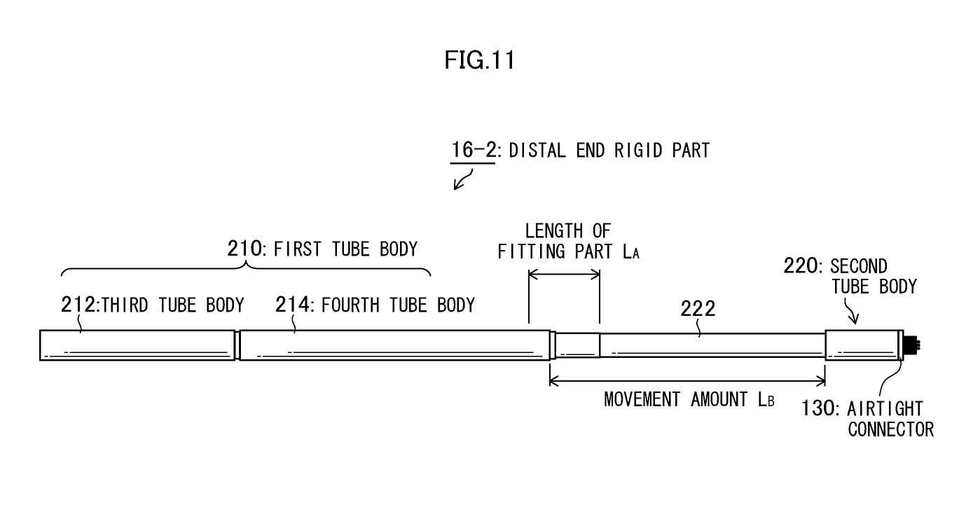

FIGS. 10 and 11 are external views showing a second embodiment of the endoscope according to the present invention in states in which the distal end rigid part 16-2 of an insertion section 12 of the endoscope 10 is contracted and that is extended, respectively.

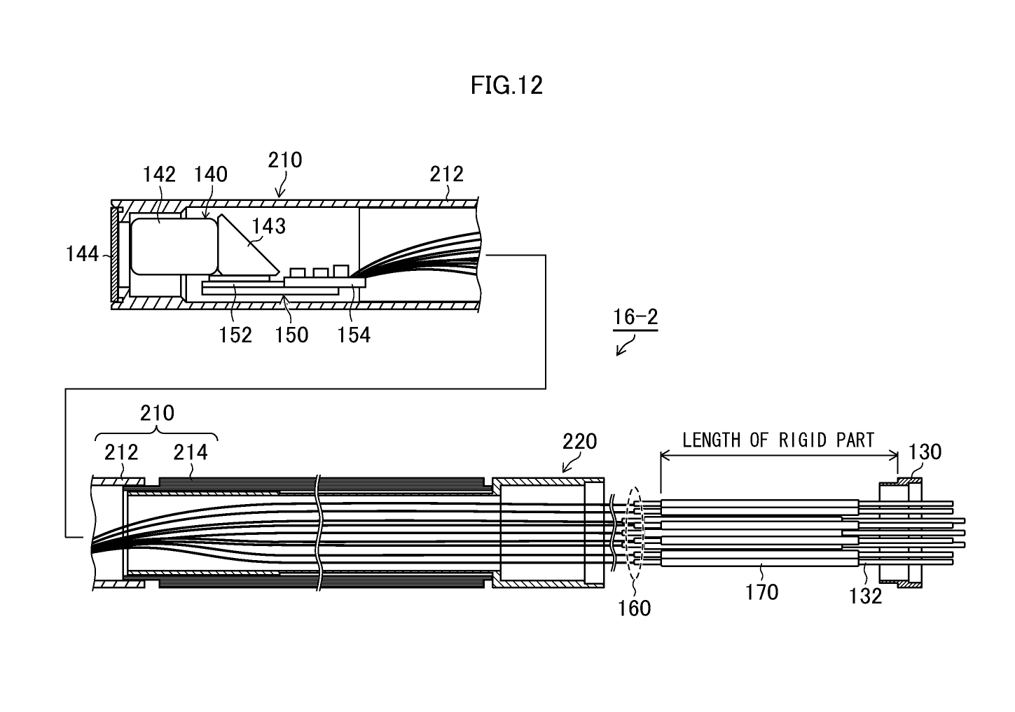

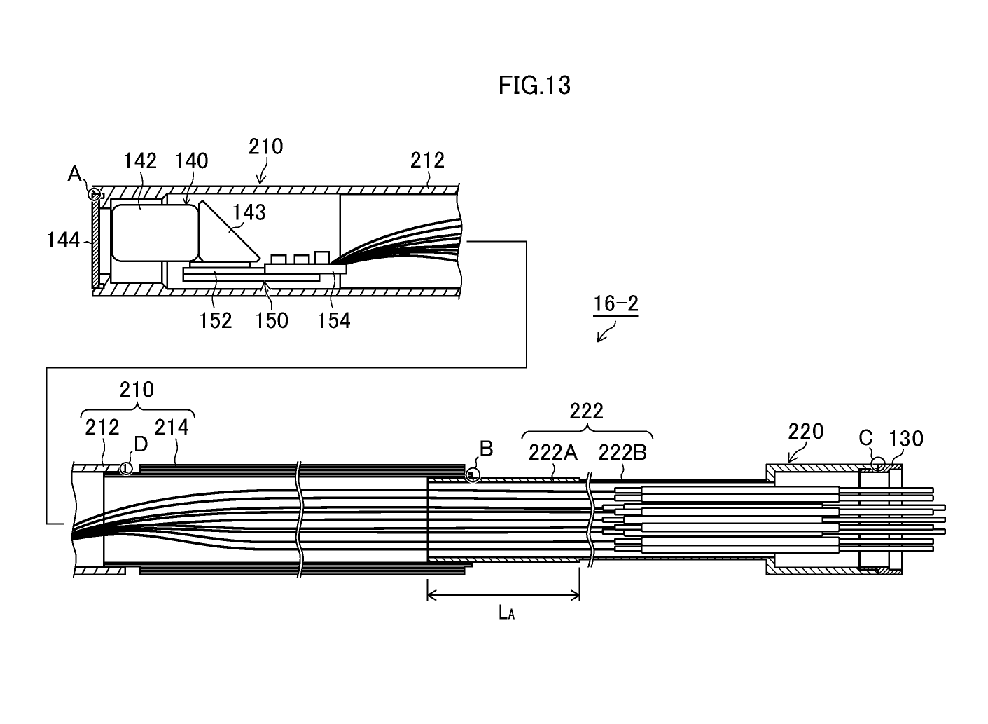

FIGS. 12 and 13 show enlarged main sections of the distal end rigid part 16-2 shown in FIGS. 10 and 11, respectively. In FIGS. 10 to 13, a part common to that in the distal end rigid part 16-1 shown in FIGS. 2 to 5 is indicated by the same reference numeral, and a detailed description of the part is omitted.

As shown in FIGS. 10 to 13, the distal end rigid part 16-2 is mainly composed of a first tube body 210, a second tube body 220 that is joined to the first tube body 210, and the airtight connector 130.

The first tube body 210 is composed of a third tube body 212, and a fourth tube body 214 that is joined to the third tube body 212.

In the third tube body 212, as shown in FIGS. 12 and 13, the optical unit 140 and the imaging device unit 150 are stored. The first tube body 210 is divided into the third tube body 212 and the fourth tube body 214, and a length of the third tube body 212 is made shorter. This makes it possible to facilitate an operation of inserting the optical unit 140 and the imaging device unit 150 from a proximal end part side of the third tube body 212 and fastening them in the third tube body 212.

If the fourth tube body 214 is lengthened enough, it is possible to secure a movement amount in which the fourth tube body 214 and the second tube body 220 can be moved while relatively sliding them to each other.

After the optical unit 140 and the imaging device unit 150 are inserted and fixed in the third tube body 212, a distal end part of the fourth tube body 214 is fitted to the proximal end part of the third tube body 212 to constitute the first tube body 210. The first tube body 210 corresponds to the first tube body 110 shown in FIGS. 2 to 5, and is longer than the first tube body 110.

The second tube body 220 includes a sliding part 222 as shown in FIGS. 11 and 13. The sliding part 222 has a fitting part 222A that is slidably fitted in an inner circumferential surface of the fourth tube body 214 with a fixed gap, and a reduced diameter part 222B having an outer diameter slightly smaller than that of the fitting part 222A. The second tube body 220 having the sliding part 222 is capable of moving (sliding) inside the fourth tube body 214 within a range of a maximum movement amount LB shown in FIG. 11.

It is required that a length L_{A} of the fitting part 222A is set at an appropriate length.

If the length L_{A} of the fitting part 222A is too long, sliding resistance increases to make the movement difficult. On the other hand, if the length L_{A} of the fitting part 222A is too short, as shown in FIG. 13, when the second tube body 220 is moved so that the second tube body 220 and the airtight connector 130 are fitted to each other, a proximal end part of the first tube body 210 (fourth tube body 214) and the reduced diameter part 222B of the second tube body 220 are overlapped with each other. As a result, a gap occurs between the inner circumferential surface of the proximal end part of the fourth tube body 214 and an outer circumferential surface of the reduced diameter part 222B. The gap is obstruction when laser welding is performed on the proximal end part of the first tube body 210 and a distal end part of the second tube body 220.

The movement amount La of the second tube body 220 varies as shown in FIGS. 10 and 11 due to a variation and the like of the extra length of the cables 160 extended from the proximal end part of the second tube body 220 as shown in FIG. 10. The length L_{A} of the fitting part 222A is secured to allow the fitting part 222A of the second tube body 220 to be fitted at a position of an end face of the proximal end part of the first tube body 210 even in the case above. That is, it is preferable that the length L_{A} of the fitting part 222A is shorter than a length in which movement is difficult due to sliding resistance as well as is a length in which the extra length of the cables 160 is adjustable.

As shown in FIG. 10, the distal end rigid part 16-2 of the present embodiment is capable of exposing the cables 160 with a length enough for the connecting work between the cables 160 and the airtight connector 130 by moving the second tube body 220 so that the sliding part 222 (FIG. 11) of the second tube body 220 is stored in the first tube body 210 (fourth tube body 214).

As illustrated in FIG. 9, a core wire of one end of the exposed cable 160 and the pin 132 of the airtight connector 130 are electrically connected through the pipe-shaped conductive member 170 (FIG. 12).

It is preferable that the maximum movement amount LB in which the first tube body 210 (fourth tube body 214) and the second tube body 220 are relatively movable is twice or more a length (a length of the rigid part) from the airtight connector 130 (a contact face of the flange part 130c) to an end part of each of the conductive members 170 connected to the airtight connector 130 on a side connected to each of the cables. As a result, it is possible to facilitate the connecting work between the cables 160 and the airtight connector 130.

When the connecting work between the cables 160 and the airtight connector 130 is finished, the first tube body 210 and the second tube body 220 are relatively moved so that the airtight connector 130 is fitted to the proximal end part of the second tube body 220 as shown in FIGS. 11 and 13.

Subsequently, as shown in FIG. 13, a fitting part A of the optical window 144 provided with a surface on which a metal film is evaporated and the distal end of the third tube body 212 is scaled by soldering.

Next, the third tube body 212 and the fourth tube body 214 are joined in an airtight manner. In the embodiment, a fitting part D (whole circumference of a proximal end part of the third tube body 212) in which the proximal end part of the third tube body 212 and the distal end part of the fourth tube body 214 are fitted is sealed by laser welding.

Next, the fourth tube body 214 and the second tube body 220 are joined in an airtight manner. In the embodiment, a fitting part B (whole circumference of a proximal end part of the fourth tube body 214) in which the proximal end part of the fourth tube body 214 and the distal end part of the second tube body 220 are fitted is sealed by laser welding.

Finally, the proximal end part of the second tube body 220 and the airtight connector 130 are joined in an airtight manner. In the embodiment, a fitting part C (whole circumference of the proximal end part of the second tube body 220) in which the proximal end part of the second tube body 220 and the airtight connector 130 are fitted is sealed by laser welding.

Accordingly, it is possible to maintain airtightness of the inside of the distal end rigid part 16-2 (the first tube body 210 (the third tube body 212+the fourth tube body 214) and the second tube body 220) storing the optical unit 140 and the imaging device unit 150, so that the endoscope provided with the distal end rigid part 16-2 can cope with autoclave sterilization.

Order of sealing operations in four fitting parts A, B. C, and D is not limited to the order described above. The sealing operations for the fitting part A between the optical window 144 and the distal end of the third tube body 212, and for the fitting part D between the proximal end part of the third tube body 212 and the distal end part of the fourth tube body 214, may be performed before the connecting work between the cables 160 and the airtight connector 130.

[Others]

Although the first and second tube bodies and the like of the present embodiment have a cylindrical shape, the bodies are not limited to the cylindrical shape, but may have a cross section formed into a shape in which a part of a circle is linear such as a D-shape, or may be a polygon tube and the like. The slide part between the first and second tube bodies is provided in the second tube body side, but it may be provided in the first tube body side.

In addition, in the present embodiment, although the endoscope having the imaging device unit of a transversely mounted type is described, the present invention is also applicable to an endoscope having an imaging device of a longitudinally mounted type.

Further, in the present embodiment, although the endoscope applied to surgery is described, the present invention is not limited to a type of an endoscope, but is applicable to various endoscopes, such as a transnasal endoscope, a colonoscope, and an industrial endoscope.

The present invention is not limited to the embodiments described above, and therefore it is needless to say that a variety of modifications is possible within a range without departing from the spirit of the present invention.

What is claimed is:

1. An endoscope comprising:
a first tube body that is provided with an optical window joined to a distal end thereof in an airtight manner, and that stores an optical unit and an imaging device unit;
a second tube body that is movable by sliding on an inner circumferential surface or an outer circumferential surface of the first tube body; and
an airtight connector that is joined to a proximal end part of the second tube body in an airtight manner, and that is connected to cables extending from the imaging device unit, wherein
the first tube body and the second tube body are joined together in an airtight manner after at least the cables and the airtight connector are connected.

2. The endoscope according to claim 1, wherein the cables have a length corresponding to a distance from the imaging device unit to the airtight connector.

3. The endoscope according to claim 1, wherein
one of the first tube body and the second tube body has a fitting part in which the other of the first tube body and the second tube body is fitted with a fixed gap, and
the fitting part has a length by which an extra length of the cables is adjustable.

4. The endoscope according to claim 1, wherein
the airtight connector includes a plurality of pins penetrating through a connector body in an airtight manner, and
the cables and the airtight connector are electrically connected through pipe-shaped conductive members in which the pins are fitted.

5. The endoscope according to claim 4, wherein a maximum movement amount by which the first tube body and the second tube body are relatively movable is twice or more as large as a length of a rigid part of the pins of the airtight connector and the conductive members.

6. The endoscope according to claim 1, wherein the optical unit includes a folded optical system, and the imaging device unit is arranged to be parallel to a longitudinal direction of the first tube body.

7. The endoscope according to claim 1, wherein the first tube body includes:
a third tube body that is provided with the optical window fixed to a distal end thereof in an airtight manner, and that stores the optical unit and the imaging device unit; and
a fourth tube body that is joined to the third tube body in an airtight manner.

8. A method for manufacturing an endoscope comprising:
preparing a first tube body storing an optical unit and an imaging device unit, a second tube body movable by sliding on an inner circumferential surface or an outer circumferential surface of the first tube body, and an airtight connector to which cables extending from the imaging device unit are connected;
extending the cables from a proximal end part of the second tube body by relatively moving the first tube body and the second tube body so that a length from a distal end of the first tube body to the proximal end part of the second tube body is shortened;
electrically connecting the cables extending from the proximal end part of the second tube body and the airtight connector;
engaging the airtight connector with the proximal end part of the second tube body by relatively moving the first tube body and the second tube body so that the length from the distal end of the first tube body to the proximal end part of the second tube body is increased after the cables and the airtight connector are connected;
joining the optical window to the distal end of the first tube body in an airtight manner;
joining the first tube body and the second tube body in an airtight manner; and
joining the proximal end part of the second tube body and the airtight connector in an airtight manner.

9. The method for manufacturing an endoscope according to claim 8, wherein
after the cables and the airtight connector are connected, the first tube body and the second tube body are relatively moved so that the length from the distal end of the first tube body to the proximal end part of the second tube body is increased, and the airtight connector is joined to the proximal end part of the second tube body, and
the cables have a length corresponding to a distance from the imaging device unit to the airtight connector at a time when the airtight connector is joined to the proximal end part of the second tube body.

10. The method for manufacturing an endoscope according to claim 8, wherein
the airtight connector includes a plurality of pins penetrating through a connector body in an airtight manner, and
in electrically connecting the cables and the airtight connector, the cables and the airtight connector are electrically connected through pipe-shaped conductive members in which the pins are fitted.

11. The method for manufacturing an endoscope according to claim 10, wherein a maximum movement amount by which the first tube body and the second tube body are relatively movable is twice or more as large as a length from the airtight connector to an end part of each of the conductive members connected to the airtight connector on a side connected to each of the cables.

12. The method for manufacturing an endoscope according to claim 8, wherein
the first tube body includes a third tube body and a fourth tube body, and
the method further comprises:
storing the optical unit and the imaging device unit in the third tube body;
fitting the fourth tube body in the third tube body; and
joining the third tube body and the fourth tube body in an airtight manner.
